(19) United States
(12) Patent Application Publication
(10) Pub. No.: US 2024/0277463 A1
(43) Pub. Date: Aug. 22, 2024

(54) THREE-DIMENSIONAL KEYHOLE REINFORCEMENT MESH FOR PARASTOMAL HERNIA AND OSTOMY PROLAPSE PREVENTION AND REPAIR

(71) Applicant: Zhongqiu Zhang, Middlebury, CT (US)

(72) Inventor: Zhongqiu Zhang, Middlebury, CT (US)

(21) Appl. No.: 18/650,677

(22) Filed: Apr. 30, 2024

Related U.S. Application Data
(63) Continuation-in-part of application No. 18/479,023, filed on Sep. 30, 2023.
Application No. 18/479,023 is a continuation of application No. 17/356,125, filed on Jun. 23, 2021, now Pat. No. 11,779,448.

(51) Int. Cl. A61F 2/00

(52) U.S. Cl. CPC A61F 2/0063; A61F 2002/0068

(57) ABSTRACT

Pre-formed mesh configurations for repair/prevention of a parastomal hernia during ostomy treatment are disclosed. More specifically, pre-molded meshes for Sugarbaker and keyhole technique repairs have unique shapes to provide support to the abdominal wall adjacent a stoma formed during an ostomy procedure to prevent/repair formation of a parastomal hernia.

### CROSS-REFERENCE TO RELATED APPLICATIONS

This application is a continuation-in-part of U.S. application Ser. No. 18/479,024, filed Sep. 30, 2023, which is a continuation of U.S. application Ser. No. 17/356,125, filed Jun. 23, 2021, now U.S. Pat. No. 11,779,448 issued Oct. 10, 2023.

### BACKGROUND OF THE DISCLOSURE

The present disclosure relates generally to mesh structure for the prevention/repair of a parastomal hernia and/or ostomy prolapse during ostomy treatment. More specifically, the present disclosure relates to a pre-molded mesh having a unique shape to provide support to the abdominal wall adjacent a stoma formed during an ostomy procedure to prevent/repair formation of a parastomal hernia and/or ostomy prolapse. The pre-molded mesh provides a clearance channel through which the bowel can pass and fixation of the bowel in a manner that maintains proper blood flow and prevents collapse or obstruction of the exposed segment of the bowel.

A stoma is a surgical hole formed in the abdomen for a colostomy or ileostomy appliance. A parastomal hernia occurs when the intestines press outwardly against the abdominal wall surrounding the stoma or through the stoma opening. This results in the formation of a bulge under the skin that can also cause pain and bothersome leakage. A prolapse occurs when the intestines push linearly outwardly through the stoma creating an inversion of the intestinal wall.

Parastomal hernias and ostomy prolapses are the most common complication of ostomy surgery. Between 87,000 and 135,000 intestinal stomas are formed in patients each year. The fundamental problem is that the ostomy creation introduces a defect into the abdominal wall for which no healing is expected and the parastomal hernia forms because the abdominal wall is continually stretched by forces tangential to its circumference. In approximately half of ostomy patients, parastomal hernias develop as a complication that often requires surgical repair.

While stoma closure is the best option, closure is often not feasible when the stoma is needed to maintain ostomy treatment. Primary suture repair of the hernia site adjacent the stoma has an unacceptably high recurrence rate and stoma re-siting often results in three separate hernias, with a hernia at the previous stoma site, one at the midline incision and one at the new stoma site.

As a result, mesh repair has become a frequently utilized solution, where an onlay mesh approach is the primary technique. The onlay approach often allows for easy hernia reduction but may be associated with difficult adhesiolysis and results in a large peri-wound cavity. Wound complications are a concern and post-operative stoma care can be difficult for the patient to maintain resulting in increased infection risk. A midline approach to an onlay repair may be difficult in an obese patient and there may be devascularization due to creation of large tissue flaps.

The most common mesh repairs are done in the sublay and intraperitoneal positions that place the mesh below the anterior fascia. The advantage of a sublay repair is that it is performed in a sterile environment with a decreased risk of wound infection. Sublay and intraperitoneal placement of the mesh provides more biomechanical support due to the abdominal pressure further securing the mesh to the abdominal wall. While the sublay repair protects the mesh from interaction with abdominal organs, the intraperitoneal position poses an increased risk for bowel erosion and adhesion formation. In the intraperitoneal repair, care must be taken to maximize tissue apposition between the mesh and the abdominal wall to minimize the formation of seroma. This includes liberal use of closed suction drains placed between the mesh and the abdominal wall. In the sublay approach, alterations must be made to the basic technique to accommodate the stoma.

There are two primary techniques for intraperitoneal mesh repair, the Sugarbaker technique and the Keyhole technique. In the Sugarbaker technique, after the hernia is reduced and the stoma trephine reduced to appropriate size, the ostomy opening is covered with an intraperitoneally placed prosthetic mesh fabric that is sutured to the fascia. The mesh fabric is continuous generally circular piece of material with no cuts or openings. The bowel is lateralized and secured between the mesh and the peritoneum, thereby lateralizing the forces which press the bowel ventrally onto the abdominal wall. Complications of this procedure include bowel obstruction secondary to dense adhesions, wound infection, seroma formation, and pain at the site of transfascial sutures.

The other primary option for surgical repair is the Keyhole technique. In the keyhole technique, a circular two-dimensional (2D) planar cut-out of mesh is made to circumferentially surround the ostomy and cover the entire hernia defect. The mesh material has a central opening to form an annular reinforcing ring secured to the abdominal wall and a radial slit to allow the annular mesh ring to be placed around the intestine without having to disconnect the intestine from the abdominal wall. One of the tricks of this technique is to not make the keyhole too small so as to cause a bowel obstruction, but to not make it so large as to increase the risk of herniation.

It is generally known that the continuous piece of mesh utilized in the Sugarbaker repair is more reliable than the keyhole repair with a cut mesh in that hernia recurrence with a Sugarbaker repair is 12% while a keyhole repair results in a recurrence rate of around 35%.

In order to reduce the number of recurrences overall, there is a movement towards prophylactic placement of reinforcement mesh to prevention of the formation of parastomal hernias during the initial ostomy treatment. However, issues still arise revolving around the technique as well as type and size of mesh, what size hole, and how should it best be secured in place. Utilizing the existing techniques is still seen to result in collapsing of the bowel with resulting obstructions as well as mesh migration.

### SUMMARY OF THE DISCLOSURE

There is therefore a need for a repair mesh for use in repairing/preventing a parastomal hernia during ostomy treatment. There is a further need for a pre-molded repair mesh for use in repairing/preventing a parastomal hernia during ostomy treatment that resists migration and allows placement without risking collapse or blockage of the extended bowel section.

In this regard, the present disclosure provides a mesh for repair/prevention of a parastomal hernia during ostomy treatment.

More specifically, a pre-molded, three-dimensional (3D) mesh having a unique shape to provide support to the abdominal wall adjacent a stoma formed during an ostomy procedure to prevent/repair formation of a parastomal hernia is disclosed which also provides a clearance channel through which the bowel can pass in a manner that prevents collapse or obstruction of the exposed segment of the bowel.

In accordance with a first embodiment, a prosthesis mesh patch is disclosed as an improvement for the Sugarbaker technique. In the Sugarbaker technique, the parastomal hernia repair is performed via a laparotomy. The herniated opening in the abdominal wall is covered with an intraperitoneally placed prosthetic mesh that is sutured to the fascial edge. The bowel is lateralized, passing from the hernia sac between the abdominal wall and the prosthesis into the peritoneal cavity. It is known from incisional hernia repair, that an overlap of 2.5-5 cm between the mesh and the adjacent fascia is mandatory to prevent recurrent hernias. Therefore, the Sugarbaker technique was modified around the trephine opening to guarantee an adequate overlap between the mesh and the fascia.

It is of utmost importance to prevent narrowing of the bowel in the tunnel and angulation of the bowel when entering the abdominal cavity and the hernia sac. In this regard, the region to be repaired is covered with a flat mesh patch. As the flat mesh is placed it must be shaped and bent as it is stitched to the trephine to prevent its normally flat nature from compressing the bowel and causing obstruction.

In accordance with the present disclosure, a novel mesh repair prosthetic is provided that is uniquely shaped during manufacture or prior to the procedure. The mesh is preformed, or heat molded to create a raised central region that creates a preformed tunnel on the distal surface of the mesh. The tunnel in turn serves to assist in correctly positioning the repair prosthesis over the lateralized bowel as well as creating the needed clearance for the bowel to pass through the stoma without compression or obstruction thereof.

In accordance with a second embodiment, a prosthetic mesh is disclosed to improve keyhole hernia prevention/repair procedures. In the keyhole technique, the elevated bowel is passed through a central hole and a planar 2D annular keyhole mesh fabric is affixed to the abdominal wall to provide support. The present disclosure provides a preformed three-dimensional (3D) parastomal hernia reinforcement prosthetic having a generally annular flange portion with an upper surface and a lower surface and a central opening, and further having a cylindrical collar portion extending downwardly from the lower surface of the annular flange portion about the central opening. The mesh sheet further includes a keyhole slot extending radially through the annular flange portion and longitudinally through the cylindrical collar portion so that the entire prosthetic can be spread open about the slot and placed around the bowel segment. The mesh sheet still further includes opposing support walls which depend downwardly and outwardly from the lower surface of the annular flange portion and the outer sidewall of the cylindrical collar portion extending along the opposing slot edges of the keyhole slot.

In use, the cylindrical collar portion is configured to be positioned about the bowel segment secured to the stomal opening in the abdominal wall. The annular flange portion is configured to be positioned adjacent to and fastened to the abdominal wall surrounding the stomal opening. Once located in place, the support walls can be squeezed together in facing relation to close the mesh around the bowel segment and secured together with a securing element such as a surgical clip, staple or suture.

It is therefore an object of the present disclosure to provide repair meshes for use in repairing/preventing a parastomal hernia during ostomy treatment. It is a further object of the present disclosure to provide a pre-molded repair mesh for use in repairing/preventing a parastomal hernia during ostomy treatment that resists migration and allows placement without risking collapse or blockage of the extended bowel section.

It is still a further object of the present disclosure to provide a pre-molded repair mesh for use in repairing/preventing a parastomal hernia during ostomy treatment reduces the time previously needed to form the mesh as it is installed during the procedure that further assists in prosthesis placement without risking collapse or blockage of the extended bowel section.

These together with other objects of the invention, along with various features of novelty which characterize the invention, are pointed out with particularity in the claims annexed hereto and forming a part of this disclosure. For a better understanding of the invention, its operating advantages and the specific objects attained by its uses, reference should be had to the accompanying drawings and descriptive matter in which there is illustrated a preferred embodiment of the invention.

### BRIEF DESCRIPTION OF THE DRAWING FIGURES

In the drawings which illustrate the best mode presently contemplated for carrying out the present invention:

FIG. 1 is a proximal view of a first embodiment of a mesh repair prosthesis for use in a Sugarbaker technique in accordance with the present disclosure;

FIG. 2 is a proximal view of a mesh repair prosthesis positioned over the lateralized bowel adjacent the stomal opening in accordance with the present disclosure;

FIG. 3 is a lateral perspective view of a Sugarbaker mesh repair prosthesis positioned over the lateralized bowel adjacent the stomal opening in accordance with the present disclosure;

FIG. 4 is a partial cross-sectional view of a mesh repair prosthesis positioned over the lateralized bowel adjacent the stomal opening in accordance with the present disclosure;

FIG. 5 is a perspective view of a second embodiment of a mesh repair prosthesis for use in a keyhole technique in accordance with the present disclosure;

FIG. 6 is another perspective view thereof;

FIG. 7 is still another perspective view thereof with added dimensional ranges for the flange and collar;

FIG. 8 is a lateral perspective view of a keyhole mesh repair prosthesis positioned around the bowel adjacent the stomal opening in accordance with the present disclosure; and

FIG. 9 is a partial cross-sectional view of the keyhole mesh repair prosthesis positioned around the bowel adjacent the stomal opening in accordance with the present disclosure.

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

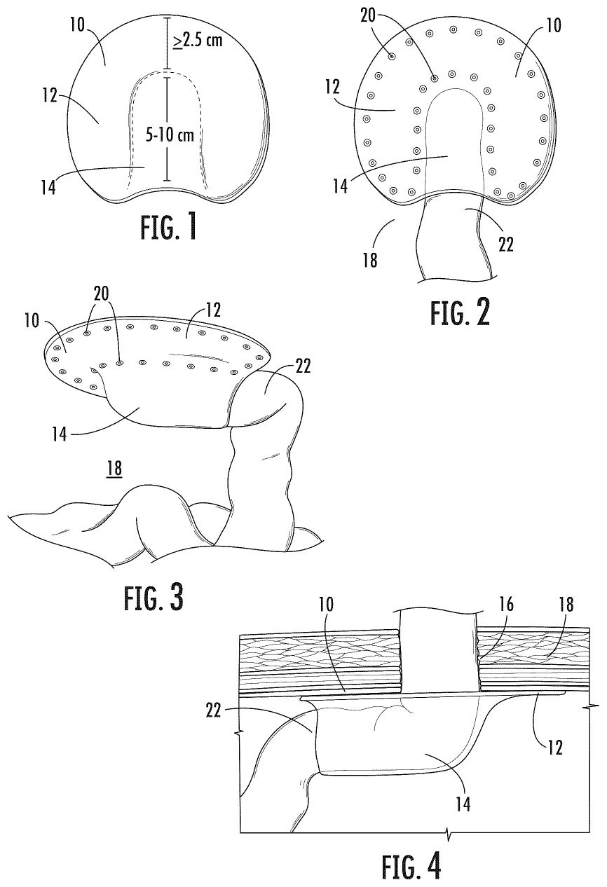

Now referring to the drawings, the preformed prosthetic repair mesh for a Sugarbaker parastomal hernia repair is shown and generally illustrated in FIG. 1-4. As can be seen in FIG. 1, a preformed mesh prosthetic 10 is disclosed for repair/prevention of a parastomal hernia during ostomy treatment. More specifically, the preformed mesh prosthetic 10 has a unique shape including blunted or rounded edges to improve placement and increase the biocompatibility of the preformed mesh prosthetic 10. The includes a margin region 12 that is preferably greater than or equal to 2.5 cm in width to provide support to the abdominal wall adjacent a stoma formed during an ostomy procedure to prevent/repair formation of a parastomal hernia and to provide sufficient overlap onto the adjacent fascia to provide effective repair and prevent formation of additional hernias.

In addition, the preformed mesh prosthetic 10 includes a preformed clearance channel 14 through which the bowel can pass in a manner that prevents collapse or obstruction of the exposed segment of the bowel. The preformed clearance channel 14 is formed in the preformed mesh prosthetic 10 having a size and shape to accommodate the protrusion of the lateralized bowel such that support is provided to the facia adjacent the stoma and yet no pressure is exerted onto the bowel thereby preventing collapsing or obstruction of the bowel that passes therethrough. The extension length of the preformed clearance channel 14 is preferably between 5 cm and 10 cm in length and has a smooth rounded shape and transitions so as to improve placement and increase the biocompatibility of the preformed mesh prosthetic 10.

Turning now to FIGS. 2-4, in accordance with the present disclosure, the preformed mesh prosthetic 10 is disclosed as a modification for the Sugarbaker repair technique. In the Sugarbaker technique the parastomal hernia repair is performed via a laparotomy. The herniated region adjacent the stomal opening 16 in the abdominal wall 18 is covered with an intraperitoneally placed preformed mesh prosthetic 10 that is attached to the fascial edge using sutures. The preformed mesh prosthetic 10 may include preformed openings 20 to facilitate placement of sutures or surgical tacks for fastening to the trephine, further the preformed openings may further include reinforcement to increase the durability of the preformed mesh prosthetic 10. The openings are preferably positioned about the outer margin of the preformed mesh prosthetic 10 and at the transition between the margin region 12 and the preformed clearance channel 14.

The lateralized bowel 22, passes from the hernia sac between the abdominal wall and the preformed mesh prosthetic 10 into the peritoneal cavity. As stated previously, it is known from incisional hernia repair, that an overlap of 2.5-5 cm between the preformed mesh prosthetic 10 and the adjacent abdominal wall 18 fascia is mandatory to prevent recurrent hernias.

Given that it is of utmost importance to prevent narrowing of the bowel in the tunnel and angulation of the bowel when entering the abdominal cavity and the hernia sac, the region to be repaired is covered with the preformed mesh prosthetic 10. In the prior art the relatively stiff and rigid mesh is flat and it must be shaped and bent as it is stitched to the trephine to prevent its normally flat nature from compressing the bowel and causing obstruction.

In accordance with the present disclosure, a preformed mesh prosthetic 10 is provided that is shaped during manufacture or prior to the procedure. The preformed clearance channel 14, in turn, serves to assist in correctly positioning the preformed mesh prosthetic 10 over the lateralized bowel as well as creating the needed clearance for the bowel to pass through the stoma without compression or obstruction thereof.

The preformed mesh prosthetic 10 of the present disclosure is preferably formed to have a mesh matrix using a material selected from the group consisting of the following materials: synthetic mesh such as polypropylene, polyester, expanded polytetrafluoroethylene (ePTFE); bioabsorbable; bio-sourced tissue or combinations thereof. Further, the mesh may be coated with absorbable fatty acids, cellulose or collagen to prevent adhesions thereto. The unique shape of the preformed mesh prosthetic 10 is preferably preformed during the mesh manufacture either by shaping the mesh as it is formed/woven or by heat molding the mesh in a separate manufacturing step. Further, the preformed mesh prosthetic 10 may be shaped using heat molding by the surgeon prior to the procedure.

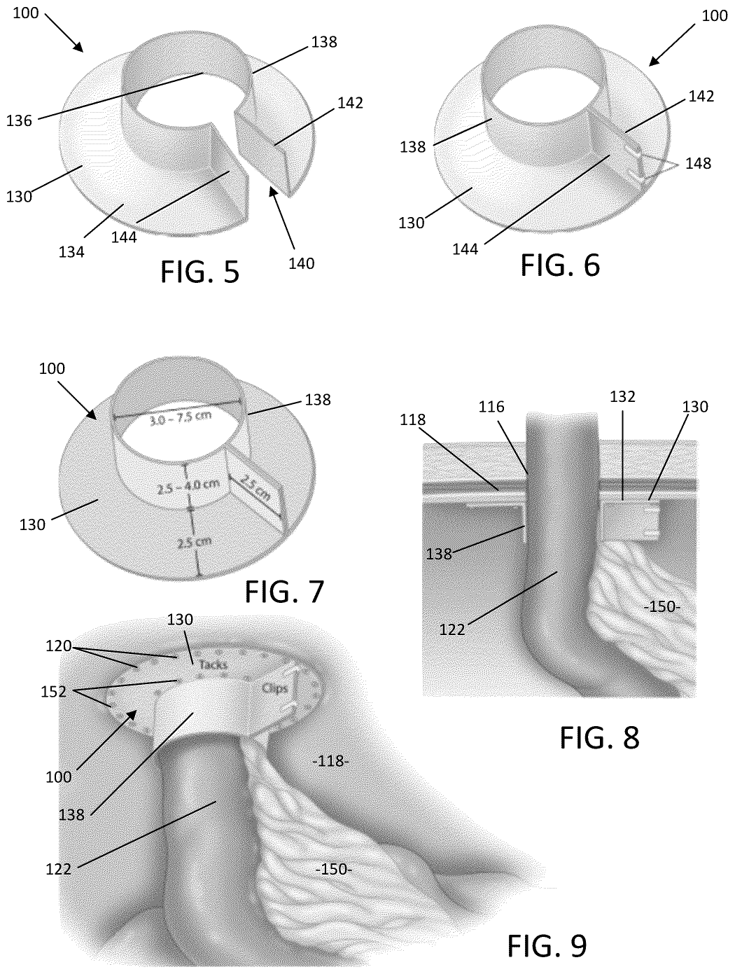

Turning now to FIGS. 5-9, a second embodiment of the pre-formed mesh is illustrated and generally indicated at 100. The prosthetic mesh 100 is disclosed as an improvement to a keyhole hernia prevention/repair procedure. In a conventional keyhole technique, the elevated bowel 122 is passed through an abdominal stoma opening 116 and a planar, annular keyhole mesh fabric (not shown) is positioned around the bowel and affixed to the abdominal wall to provide support.

The present disclosure provides a preformed parastomal hernia reinforcement prosthetic 100 having a generally annular flange portion 130 with an upper surface 132 and a lower surface 134 and a central opening 136, and further having a cylindrical collar portion 138 extending downwardly from the lower surface 134 of the annular flange portion 130 about the central opening 136. The mesh sheet 100 further includes a keyhole slot 140 extending radially through the annular flange portion 130 and longitudinally through the cylindrical collar portion 138 so that the entire prosthetic 100 can be spread open about the slot 140 and placed around the bowel segment 122. The mesh sheet 100 still further includes opposing support walls 142, 144 which depend downwardly from the lower surface 134 of the annular flange portion 130 and outwardly from the outer surface of the cylindrical collar portion 138 extending along the opposing slot edges of the keyhole slot 140. The support walls 142, 144 provide a measure of rigidity and form to the mesh prosthetic 100 to maintain its shape.

Referring to FIG. 7, the annular flange portion 130 may have a flange width of about 2.5 cm or greater to provide sufficient abdominal wall overlap to prevent recurrent hernias. The cylindrical collar portion 138 may have a diameter of about 3.0 or greater and more preferably between about 3.5 cm and 7.5 cm. The cylindrical collar portion 138 may further have an axial length of about 2.5 cm or greater, and more preferably between about 2.5 cm and about 4.0 cm.

In use, the cylindrical collar portion 138 is configured to be positioned about the bowel segment 122 which is secured to the stomal opening 116 in the abdominal wall 118. The annular flange portion 130 is configured to be positioned adjacent to and fastened to the abdominal wall 118 surrounding the stomal opening 116. The mesh prosthetic is rigid enough to retain its shape, but still flexible in order to be spread open to fit around the bowel segment 122 for placement.

Once located in place, the support walls 142, 144 can be squeezed together in facing relation (see FIGS. 6 and 7) to close the prosthetic around the bowel segment 122, and are then secured together with a securing element 148 such as a surgical clip, staple or suture.

The size and semi-rigid shape of the prosthetic 100 both supports the bowel 122 and provides sufficient space for the menditary tissue 150 which supplies blow flow to the intestinal tissues.

The preformed mesh prosthetic 100 may include preformed openings 120 to facilitate placement of sutures or surgical tacks 152 for fastening to the trephine. The preformed openings 120 may be positioned about both the inner and outer peripheral margins of the annular flange portion and may further include reinforcement to increase the durability of the preformed mesh prosthetic 100.

Similar to the above Sugarbaker mesh 10, the preformed keyhole mesh prosthetic 100 may also be shaped during manufacture or prior to the procedure and may be preferably formed to have a mesh matrix using a material selected from the group consisting of the following materials: synthetic mesh such as polypropylene, polyester, expanded polytetrafluoroethylene (ePTFE); bioabsorbable; bio-sourced tissue or combinations thereof.

Further, the mesh 100 may also be coated with absorbable fatty acids, cellulose or collagen to prevent adhesions thereto. The unique shape of the preformed mesh prosthetic 100 is preferably preformed during the mesh manufacture either by shaping the mesh as it is formed/woven or by heat molding the mesh in a separate manufacturing step. Further, the preformed mesh prosthetic 10 may be shaped using heat molding by the surgeon prior to the procedure.

It can therefore be seen that the present disclosure provides unique repair meshes for use in repairing/preventing a parastomal hernia during ostomy treatments. The present disclosure further provides pre-molded repair meshes for use in repairing/preventing a parastomal hernia during ostomy treatment that resists migration and allows placement without risking collapse or blockage of the extended bowel section. Still further, the present disclosure provides pre-molded repair meshes for use in repairing/preventing a parastomal hernia during ostomy treatment which reduce the time previously needed to form the mesh as it is installed during the procedure that further assists in prosthesis placement without risking collapse or blockage of the extended bowel section. For these reasons, the present disclosure is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A preformed parastomal hernia reinforcement prosthetic, comprising:
a unitary, bio-implantable, heat-formable mesh sheet having a generally annular flange portion with an upper surface and a lower surface and a central opening, and further having a cylindrical collar portion extending downwardly from the lower surface of the annular flange portion about the central opening,
said mesh sheet further including a keyhole slot extending radially through the annular flange portion and longitudinally through the cylindrical collar portion,
said mesh sheet still further including opposing support walls depending from said annular flange portion and said cylindrical collar portion and extending along opposing edges of the keyhole slot,
said cylindrical collar portion being configured to be positioned about a bowel segment secured to a stomal opening in an abdominal wall,
the annular flange portion being configured to be positioned adjacent and fastened to said abdominal wall surrounding said stomal opening; and
at least one securing element for securing the opposing support walls in facing relation.

2. The preformed parastomal hernia reinforcement prosthetic of claim 1, further comprising:
attachment openings positioned about an outer peripheral margin of said annular flange portion.

3. The preformed parastomal hernia reinforcement prosthetic of claim 1, further comprising:
attachment openings positioned about an inner peripheral margin of said annular flange portion.

4. The preformed parastomal hernia reinforcement prosthetic of claim 2, further comprising:
attachment openings positioned about an inner peripheral margin of said annular flange portion.

5. The preformed parastomal hernia reinforcement prosthetic of claim 1, wherein said annular flange portion has a width of 2.5 cm or greater.

6. The preformed parastomal hernia reinforcement prosthetic of claim 1, wherein said cylindrical collar portion has a diameter of 3.0 cm or greater.

7. The preformed parastomal hernia reinforcement prosthetic of claim 6, wherein said cylindrical collar portion has a diameter of 3.0 cm or greater.

8. The preformed parastomal hernia reinforcement prosthetic of claim 1, wherein said cylindrical collar portion has an axial length of 2.5 cm or greater.

9. The preformed parastomal hernia reinforcement prosthetic of claim 6, wherein said cylindrical collar portion has an axial length of 2.5 cm or greater.

10. The preformed parastomal hernia reinforcement prosthetic of claim 7, wherein said cylindrical collar portion has an axial length of 2.5 cm or greater.

11. The preformed parastomal hernia reinforcement prosthetic of claim 1, wherein said annular flange portion, said cylindrical collar portion and said support walls are formed in said mesh sheet during manufacture thereof.

12. The preformed parastomal hernia reinforcement prosthetic of claim 1, wherein said mesh sheet is a synthetic mesh material.

13. The preformed parastomal hernia reinforcement prosthetic of claim 1, wherein said mesh sheet is a bio-sourced mesh material.

14. The preformed parastomal hernia reinforcement prosthetic of claim 1, wherein said mesh sheet is selected from the group consisting of: synthetic mesh, composite mesh, polypropylene, polyester, ePTFE, bio-absorbable mesh, bio-sourced tissue and combinations thereof.

15. The preformed parastomal hernia reinforcement prosthetic of claim 1, wherein said mesh sheet is coated with a material selected from the group consisting of: absorbable fatty acids, cellulose, collagen and combinations thereof.
